SEQUENCE LISTING (2 sequences)

SEQ ID NO: 1 - DNA, 2745 bp, Saccharomyces cerevisiae
tcactgaaaaatatttattttttcagtgtctacttcgtggcctttgaaatgtgtagtaagcctagaccatatatcatcgtaaagatttagttcaaaatagtcgtgtataagatcgtaaagattagaaaagttcttgaaaagtgaatccgatttcattccatcaaatgcttgttgcacgatgctttcaaaaatacttctagatggatcatcaccaggagcgagcgttctgtaactcgaaaacaattggccaagctgtgttaatcgagcaggatgaatgtgcaatatattctcgaatgacgatctttcttgctgaaaagaatctatcgacagatttggcatagcatgtgcattttttggtattttctcataattgtgaaaacactgtaagtagtcgttaggtacaaattcgcaatttaaaggttgatctatgaatagtatgataaaatcagccgccccttcaaagtagtcattaaaattgggtaatatttttgtctctagaatctttagtcttcgccttatatgaaatccatttaaggcatggatcacattattcttccattgaataacaacggtcttaccatctaacgtttcataatgactgtcccttgaattcgtatttatgtaagagttttcagtcattaacaatatgtaagagttaataaactctgcattataacagaggtcatgtaacggcagtttgctcttgacatcgtggtaattcaaaaggtattctgaaggaggtgctaggaggataaacctacgggtttttaaattagcgaaaagctttttcaatggggatctttctggttcgggacagttaaatatagcattaaccagcgggttcaacagcgttggtaaatggtaaactgacattgtcaaccctagctaagccatagcttttccgttgaggaatttacaataaggtggttcctttagttataaattgcaactgccaaaattttcgggtagcggcaaaattaaacgaaaaccttctgtcatattaaacatagtttagtgacgcaatgcacacacactcatatatatgtatatatttatctatatcataggtgtaaggcaaagttatcacgtgaatatactgtttagtatacaaatacgttgatttaacggctgagagattcccaaacaaagtaacgcttagtttagctaaagtagaatcgaagcgcagtgaaggcaagacgagttatccctttatcacaaaatataaaaaataggtaaagaggggggtgggggtggttcggactttgcatctcggtgcattagtaccactcagcttcagttataggaatactgacaatgttcttgagccagggagaactgaaatttccataccctttaaccctttattcttgagcatatgatcttatttttccactcagtacaaaatacacatcccgcatatgatctggacactgaataaactaggggctttatgatggagtagtagacctggatacgtagtcaccacagggatcctaaactgcgtcatagtaagtttctttgatactagaatgtccgtctctcgtagtagttgatatcccgtatttctagttagtgacgtttataaatagggagaaaaaaaatcggagtaacatttccatgcctttttttgacacttcagaccagatacgccaccagctacaaactaacaatgtccactgatagtacacgctatccaattcagattgagaaagcctcgaatgatccaacccttctgcttaatcacacatgtttaagagtcaaggatccagcaagggctgttaagttctacaccgaacacttcggtatgaagctattaagcagaaaggattttgaagaagcaaaatttagcttgtactttttaagctttccaaaagacgacatacccaaaaataagaatggagagcctgatgtttttagcgcacacggtgtcttagaactaactcacaattggggtactgaaaaaaacccagactacaagatcaacaacgggaatgaggaacctcatcgtggatttgggcacatctgtttttctgtatccgatatcaataaaacctgcgaagagctagaatctcagggtgtcaaattcaagaagagactctctgaaggaagacagaaggacattgcgtttgctttagaccctgatggatactggattgagttgatcacatattctagagagggtcaggaatacccaaagggctcagtaggtaacaagttcaatcataccatgattcgtattaaaaacccaacccggtctttagaattctaccagaatgtgttgggaatgaaattattaagaactagtgagcacgaaagtgcaaaatttacgttatactttcttggttatggcgttccaaagaccgacagcgttttttcatgtgaaagtgtgttggagttaactcataattggggaactgagaatgatccaaacttccactatcataacggtaactcagagccccagggttatggtcacatctgcataagttgcgatgacgctggcgccctttgtaaagaaattgaagtgaaatacggcgataagatccaatggtctcctaaatttaaccaaggcagaatgaagaatattgcctttttgaaggatcctgatggttattccattgaagtcgttccttatggtttgattgcctaaaaagttgaaatgaataggaggttagtattttttttttacaataatgaccattcttgttgacttttcatacactaattattatttatattttgtat

SEQ ID NO: 2 - protein, 326 aa, Saccharomyces cerevisiae
MSTDSTRYPIQIEKASNDPTLLLNHTCLRVKDPARAVKFYTEHFGMKLLSRKDFEEAKFSLYFLSFPKDDIPKNKNGEPDVFSAHGVLELTHNWGTEKNPDYKINNGNEEPHRGFGHICFSVSDINKTCEELESQGVKFKKRLSEGRQKDIAFALDPDGYWIELITYSREGQEYPKGSVGNKFNHTMIRIKNPTRSLEFYQNVLGMKLLRTSEHESAKFTLYFLGYGVPKTDSVFSCESVLELTHNWGTENDPNFHYHNGNSEPQGYGHICISCDDAGALCKEIEVKYGDKIQWSPKFNQGRMKNIAFLKDPDGYSIEVVPYGLIA

(19) United States
(12) Patent Application Publication
(10) Pub. No.: US 2017/0191087 A1
(43) Pub. Date: Jul. 6, 2017

(54) Causative genes conferring acetic acid tolerance in yeast

(71) Applicants: VIB VZW, Gent (BE); KATHOLIEKE UNIVERSITEIT LEUVEN K.U. LEUVEN R&D, Leuven (BE)

(72) Inventors: Johan Thevelein, Blanden (BE); Maria Remedios Foulquié Moreno, Brussels (BE); Meijnen Jean-Paul, Rhenen (NL)

(21) Appl. No.: 15/314,450

(22) PCT Filed: May 26, 2015
(86) PCT No.: PCT/EP2015/061590

§ 371 Date: Nov. 28, 2016

(30) Foreign Application Priority Data
May 26, 2014 (EP) 14169848.0

(51) Int. Cl. C12P 7/06; C12P 7/10; C12N 9/88

(52) U.S. Cl. CPC C12P 7/06; C12N 9/88; C12Y 404/01005; C12P 7/10

(57) ABSTRACT

The present invention relates to the use of GLO1 to modulate acetic acid tolerance in yeast. More specifically, it relates to the use of a specific GLO1 allele to confer tolerance to acetic acid, and to improve the fermentation performance of yeast in the presence of acetic acid.

The present invention relates to the use of GLO1 to modulate acetic acid tolerance in yeast. More specifically, it relates to the use of a specific GLO1 allele to confer tolerance to acetic acid, and to improve the fermentation performance of yeast in the presence of acetic acid.

Hydrolysates of lignocellulose are an interesting source for the production of bioethanol. However, one of the problems is the presence of toxic compounds such as acetic acid, furfural and lignin derivatives. Resistance against these inhibitors is essential for an efficient bioethanol production (Olsson and Hahn-Hägerdal, 1993). Especially acetic acid is known to have an inhibitory effect (Limtong et al., 2000). However, although overexpression of a single io gene may improve acetic acid tolerance (Tanaka et al., 2012), it is important to understand the interplay of genes, proteins and other components that determine the physiological properties of a microorganism.

In the past, research focused indeed primarily on the identification of single alleles or genetic loci that are involved in physiological traits (Glazier et al., 2002). However, in contrast to Mendelian traits (traits that are caused by one single locus), quantitative traits are caused by multiple genetic loci, which makes the unraveling of these complex traits rather difficult (Steinmetz et al., 2002). In addition, the genetic mapping of quantitative trait loci (QTL) is hampered by genetic heterogeneity, variable phenotypic contributions of each QTL, epistasis and gene-environment interactions (Flint and Mott, 2001). These limitations have facilitated the development of novel technologies to simultaneously identify genomic loci that are involved in complex traits. With these technologies, phenotypes like high-temperature tolerance, efficient sporulation and chemical resistance have been genetically unraveled (Steinmetz et al, 2002; Deutschbauer and Davies, 2005; Ehrenreich et al., 2010).

Recently, Swinnen et al. (2012) developed such a strategy, which was successfully employed to identify genetic determinants that are involved in high ethanol tolerance in the yeast

Saccharomyces cerevisiae. In this strategy, called pooled-segregant whole-genome sequence analysis, it was demonstrated that QTLs underlying a complex trait can be mapped using small populations of segregants. However, the identification of causative mutations in these QTLs remains cumbersome since this method results in a relatively large size of the identified loci, which infers the analysis of a large number of genes. Reducing the size of QTLs can be achieved with inbreeding crosses, as was recently described by Parts et al (2011). However, the use of very large pools makes it an extensive procedure, especially since phenotyping industrially relevant traits often requires elaborate procedures, making the use of large numbers of segregants undesirable. Furthermore, although inbreeding crosses can be used to decrease the size QTLs, it remains unknown how it influences the mapping of minor loci.

In order to investigate the effect of inbreeding crosses on QTL mapping of industrially relevant strains, we have applied the pooled-segregant whole-genome sequencing analysis methodology on F1 and F7 segregants of a cross between a yeast strain that is superior for acetic acid tolerance and an industrial strain that is inferior for the same trait. Acetic acid tolerance is an industrially important characteristic as yeast fermentation is severely inhibited by this weak organic acid. As mentioned above, the presence of acetic acid in lignocellulosic hydrolysate strongly affects the fermentative capacity of yeast (Casey et al., 2010; Huang et al., 2011; Narendranath et al, 2001; Taherzadeh et al., 1997; Almeida et al., 2007). Especially the fermentation of pentose sugars suffers from the presence of acetic acid (Casey et al., 2010; Bellissimi et al, 2009; Matsushika and Sawayama, 2012), emphasizing the importance of high acetic acid tolerance to enable efficient conversion of all sugars in lignocellulosic hydrolysate into ethanol. However, multiple attempts to rationally engineer increased acetic acid tolerance in yeast were met with limited success as a high number of genes is involved in the response to acetic acid stress (Abott et al., 2007; Mira et al., 2010 a & b; Li and Yuan, 2010, Hasunuma et al., 2011; Zhang et al., 2011). Random approaches such as evolutionary engineering has rendered improved strains in terms of acetic acid tolerance (Koppram et al., 2012; Wright et al., 2011), but this method leads to overselection of a single trait and to possible loss of other important properties.

We found for the first time that increased recombination frequency indeed results in the expected smaller loci, but also in unexpected appearance and disappearance of QTLs, compared to QTL mapping without inbreeding crosses. Furthermore, combining individual whole-genome sequencing data of acetic acid tolerant segregants with bioinformatics analysis enabled QTL mapping to single gene level.

Surprisingly we found that a specific allele of GLO1 is needed and sufficient to confer tolerance to relatively high concentrations of acetic acid. Replacement of the inferior allele by a superior allele results in a significant improvement of the fermentation performance in presence of at least 0.5% acetic acid.

A first aspect of the invention is the use of GLO1 to modulate the acetic acid tolerance in yeast. Preferably, said use is the use of a specific allele of GLO1 to increase the acetic acid tolerance, even more preferably said specific allele is encoding SED ID No.2, even more preferably said specific allele consist of SEQ ID No.1.

In a preferred embodiment, the use according to the invention is the overexpression of the protein, encoded by the specific allele. Such overexpression can be obtained by any method known to the person, skilled in the art. As a non-limiting example, overexpression can be obtained by incorporating more than one copy of the specific allele in a strain, or by placing the coding sequence of the specific allele under control of a strong promoter. In another preferred embodiment, said use according to the invention is the replacement of an inferior allele by the allele according to the invention.

Acetic acid tolerance as used here means that the fermentation performance of the strain in presence of acetic acid is better than that of a control strain with the same genetic background, io except for the GLO1 allele. The concentration of acetic acid in the medium is at least 0.4%, preferably at least 0.5%, more preferably at least 0.6%, even more preferably at least 0.7%, most preferably at least 0.8%. An improved fermentation performance may be measured as a higher ethanol yield, a faster fermentation rate of a shorter lag phase. Preferably said improved fermentation performance is a faster fermentation rate and/or a shorter lag period.

A GLO1 allele is called a “superior GLO1 allele” herein if, in a strain with an identical background, except for the GLO1 allele, the presence of the GLO1 allele allows improved fermentation performance in the presence of at least 0.4% acetic acid in the medium as compared to a relevant control. Analogously, a GLO1 allele is termed an “inferior GLO1 allele” herein if, in a strain with an identical background, except for the GLO1 allele, the presence of the GLO1 allele results in worse fermentation performance in the presence of at least 0.4% acetic acid in the medium as compared to a relevant control. The same applies for higher concentrations of acetic acids, e.g. 0.5%, 0.6%, 0.7%, 0.8%.

Preferably, said yeast according to the invention is a xylose fermenting yeast. A xylose fermenting yeast, as used here, can be a yeast that is naturally producing ethanol on the base of xylose, or it can be a yeast that is mutated and/or genetically engineered to ferment xylose and to produce ethanol on the base of xylose. Even more preferably, said yeast is selected from the group consisting of Saccharomyces sp., Pichia sp., Candida sp., Pachysolen sp. and Spathaspora sp. Most preferably, said yeast is a Saccharomyces sp. preferably a Saccharomyces cerevisiae.

Another aspect of the invention is a recombinant yeast strain, comprising a recombinant allele encoding SEQ ID No.2. In a preferred embodiment, said recombinant yeast strain comprises a recombinant allele consisting of SEQ ID No. 1.

Still another aspect of the invention is a method to obtain an acetic acid tolerant yeast, by crossing in a superior GLO1 allele. Crossing in, as used here, can be by classical breeding, either by making a heterozygous diploid (comprising an inferior and a superior allele), of by mating and sporulation, selecting the strain comprising the superior allele. In a preferred embodiment, crossing in comprises the replacement of an inferior GLO1 allele by a superior GLO1 allele. In a preferred embodiment, said superior GLO1 allele is encoding SEQ ID No.2. Preferably, said superior GLO1 allele is consisting of SEQ ID No.1.

Still another aspect of the invention is a method for selecting acetic acid tolerant yeast, comprising the identification of the presence of a superior GLO1 allele. Said identification can be done by any method known to the person skilled in the art. Preferably, said method comprises the sequencing of the GLO1 allele. Preferably, said superior GLO1 allele is io encoding SEQ ID No.2. Even more preferably, said superior GLO1 allele is consisting of SEQ ID No.1.

### BRIEF DESCRIPTION OF THE FIGURES

FIG. 1: Small scale fermentation of RHA strains for GLO1. The sugar conversion is expressed as % weight loss.

FIG. 2: Replacement of GLO1 in ER18 by GLO1 from 16D (A) replacement of GLO1 in ER18 by a kanamycin marker; (B) removal of the kanamycin cassette (C) Tagging of GLO1 in 16D by a kanamycin cassette and isolation of the GLO1 kanamycin fragment by PCR (D) Transformation of the tagged fragment in ER18 and removal of the kanamycin cassette.

FIG. 3: Fermentation performance of the parental ER18 strain, compared with the ER18 variant in which the original inferior GLO1 allele has been replaced by a superior 16D allele

### EXAMPLES

### Material and Methods to the Examples

### Strains Used in the Study

Ethanol red is a diploid industrial strain, and was obtained from Fermentis. The strain was sporulated, and the haploid segregants ER18 and 16D have been isolated on the base of the difference in their acetic acid resistance (see Table I)

TABLE I
Stains used for RHA analysis GLO1

| Name | Genotype | Stocknumber |
|---|---|---|
| ER18 | Inferior parent | JT 24050 |
| 16D | Superior parent | JT 24211 |
| ER18 × 16D | Hybrid of ER18 and 16D | JT 24198 |
| BY4741 glo1Δ::KanMX4 | Isogenic to BY4741; except glo1 Δ::KANMX | JT_a.390 |
| 16D glo1Δ::KanMX4 colony1 | Isogenic to 16D; except glo1 Δ::KANMX | PV_T1 |
| 16D glo1Δ::KanMX4 colony2 | Isogenic to 16D; except glo1 Δ::KANMX | PV_T2 |
| ER18 glo1Δ::KanMX4 colony1 | Isogenic to ER18; except glo1 Δ::KANMX | PV_T3 |
| ER18 glo1Δ::KanMX4 colony2 | Isogenic to ER18; except glo1 Δ::KANMX | PV_T4 |
| ER18 × 16D glo1Δ::KanMX4 colony1 | Hybrid of ER18 and 16D glo1Δ::KanMX4 colony1 | PV_T5 |
| ER18 × 16D glo1Δ::KanMX4 colony2 | Hybrid of ER18 and 16D glo1Δ::KanMX4 colony2 | PV_T6 |
| ER18 glo1Δ::KanMX4 × 16D colony1 | Hybrid of 16D and ER glo1Δ::KanMX4 colony1 | PV_T7 |
| ER18 glo1Δ::KanMX4 × 16D colony2 | Hybrid of 16D and ER glo1Δ::KanMX4 colony2 | PV_T8 |

### Construction of RHA Strains

The reciprocal deletions were engineered in the haploid strains, after which the proper haploids were crossed to obtain the diploid hybrids. The haploid deletion strains were created by gene targeting in the parental strains 16D and ER18. Deletion cassettes were PCR amplified from genomic DNA of strain BY4741 glo1Δ::KANMX4 (JT_a.390), obtained from the deletion collection (Winzeler et al., 1999), and primers B-2344 and B-2345. After io transformation with the lithium acetate method (Gietz et al., 1995), transformants were selected on YPD plates containing geneticin (200 mg/l). Deletion of GLO1 was confirmed by PCR with primercouple A-3863/B-2612. Of each transformed strain, two transformants were selected and subsequently crossed with the corresponding parental strain to construct the hybrid diploid strains. Mating type of the diploids was confirmed by diagnostic PCR for the MAT locus (Huxley et al., 1990).

### Assessment of Acetic Acid Tolerance

Acetic acid tolerance in media containing acetic acid and glucose was evaluated by determination of the fermentation performance of yeast strains in small scale, near anaerobic batch fermentations. Yeast strains were pre-cultured in YPD medium (30° C., static incubation and 60 hour). After collection (1700 g, 2 minutes) and washing of the cells with Milli-Q water, cylindrical glass tubes containing 100 ml of YP medium supplemented with 4% w/v D-glucose and a range of acetic acid, adjusted to pH=4 with HCL or KOH, were inoculated at an OD600 of 0.3. The culture was agitated continuously at 120 rpm using a magnetic rod. The fermentations were performed at 30° C. The course of the fermentation was monitored by weighing the fermentation tubes at regular intervals.

### Example 1: Screening for Superior Acetic Acid Tolerance

Ethanol Red is a diploid yeast strain that is being used for bio-ethanol production at high temperatures, showing ethanol yields of up to 18%. However, the fermentation performance of this industrial yeast strain is severely affected by acetic acid, a weak organic acid present in high quantities in lignocellulosic hydrolysates. Haploid segregants were isolated from this yeast strain and scored on acetic acid tolerance by fermentation in YPD medium supplemented with various concentrations of acetic acid. It was observed that the maximum tolerance of Ethanol io Red towards acetic acid was 0.6% (v/v) in YPD medium at a pH of 4.0. However, the lag phase was significantly prolonged by adding acetic acid to the growth medium, with a lag phase of approximately 30 hours at concentrations of 0.5% and 0.6%. The haploid Ethanol Red segregant #18 (named ER18) showed similar tolerance to acetic acid and was therefore selected for further experiments.

In order to obtain a yeast strain with high acetic acid tolerance, the in-house yeast collection and the yeast collection from the Fungal Biodiversity Centre (CBS-KNAW, Utrecht, The Netherlands) were screened under acetic acid conditions. More than 1000 yeast strains were assessed, from which strain JT 22689 showed the best performance under fermentative conditions at high acetic acid concentrations, being able to ferment glucose in the presence of 0.9% acetic acid without a lag phase (not shown). Also from this strain a haploid segregant, named 16D, could be isolated that showed a similar phenotype in terms of acetic acid tolerance.

### Example 2: QTL Mapping with Pooled F1 Segregants

Mapping the genetic determinants that are responsible for the high acetic acid tolerance of 16D was initiated by crossing the haploid segregants ER18 and 16D. The resulting hybrid strain was subsequently sporulated to obtain segregants that contain a mixture of the parental genomes. Obtained segregants were subsequently screened for high acetic acid tolerance, resulting in the identification of 27 (out of 288) segregants that were able to ferment glucose in the presence of 0.9% acetic acid, which is comparable with the tolerance observed for the superior parent strain. These 27 segregants were therefore selected for pooled-segregant whole-genome sequencing analysis. Genomic DNA isolated from the two parent strains, a pool of the 27 selected segregants and a control pool of 27 randomly selected segregants was sent for custom sequencing analysis using the Illumina HiSeq2000 technology (BGI, Hong Kong, China). The sequence reads from parent strains ER18 and 16D were aligned with the reference sequence from strain S288C. A total number of 23,150 SNPs between ER18 and 16D could be identified, which were subsequently filtered according to the method described by Duitama et al. (2012). The SNP variant frequencies were calculated by dividing the number of the alternative variant by the total number of aligned reads. The calculated variant frequencies were subsequently plotted against the respective chromosomal positions. The underlying structure in the SNP variant frequencies scatterplot of a given chromosome was identified by fitting smoothing splines in the generalized linear mixed model framework, as described by Claesen et al. (2013). Variant frequencies that significantly deviate from 50% (random segregation) are indicative of genetic linkage to the phenotype.

The results from the QTL mapping show two loci on the genome with a strong linkage to the superior segregant 16D: QTL1 on chromosome XIII and a second QTL on chromosome XVI. The statistical significance of QTL1 was confirmed using the Hidden Markov Model described previously, stretching from position 181019-294166. Both QTLs were further investigated by scoring selected SNPs in the 27 individual segregants in order to precisely determine the SNP variant frequencies and the statistical significance of the genetic linkage. Using a binomial test previously described (Swinnen et al., 2012; Claessen et al., 2013), both loci were found to be statistically significant. Furthermore, the size of both QTLs could be decreased to regions stretching from roughly 224000-277000 for QTL1 on chromosome XIII, and 568000-615000 for QTL2 on chromosome XVI.

GLO1 was confirmed as causative gene for acetic acid tolerance by RHA

### Example 3: Fermentation Assay of RHA Strains

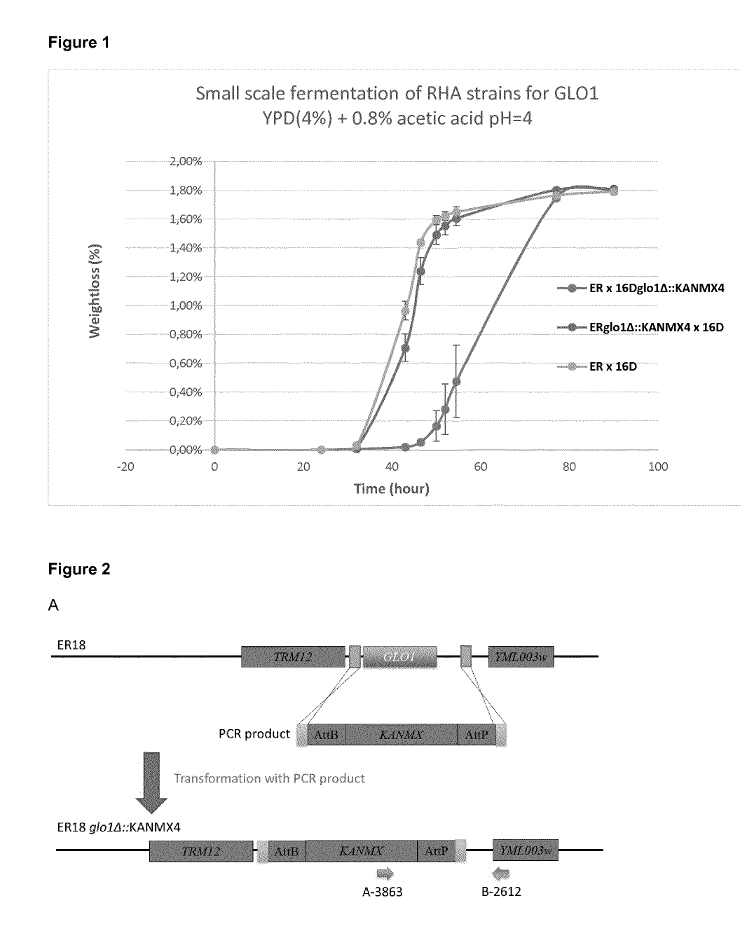

FIG. 1 shows the fermentation profiles of the RHA strains for GLO1. Every point represents the average of two biological repeats. The error bars indicate the standard error of the mean.

The strains with at least one allele originating from the 16D strain show superior fermentation performance in presence of acetic acid.

### Example 4: Replacement of the GLO1 Allele from Strain ER18 Byt the Allele From Stain 16D

In order to upgrade the GLO1 allele of ER18, a fragment comprising the ORF, 631 bp upstream and 44 bp downstream of the ORF of GLO1 was replaced by its 16D counterpart. The method to replace the allele comprises three steps:

1. Deletion of the region containing the ORF of GLO1, 631 bp upstream and 44 bp downstream in ER18. Primers B-2610 and B-2609 are used to amplify the deletion cassette from plasmid pJET1,2-AttB-KANMX-AttP.

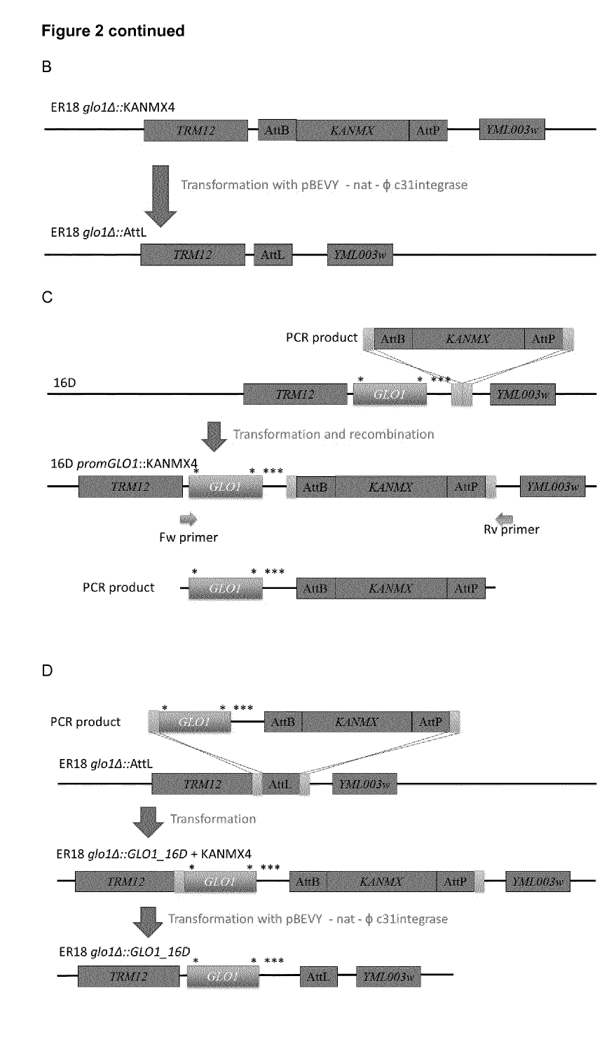

Both primers contain a 19 bp region, binding to pJET1,2-B-KANMX-P and 50 bp tails that are homologous to the nucleotides flanking the region that needs to be deleted. In the schematic representation of FIG. 2A, these homologous regions are shown as light grey boxes. After transformation, colonies will be selected on YPD plates containing geneticin (200mg/I). Hereafter, colonies were confirmed by PCR with primer couple A-3863/B-2612.

Hereafter, strain ER18 glo1Δ::KANMX4 was transformed with plasmid pBEVY-nat-Phic31integrase. After selection on YPD plates containing nourseothricin (100 mg/l), the kanamycin marker was removed due to the action of the phage derived phiC31 integrase, leaving an AttL sequence at the recombination site (FIG. 2B).

After confirming of the loss of the KANMX marker by checking the lack of growth on YPD geneticin plates, the strain was cured of the plasmid by growing several rounds in liquid YPD medium.

2. Next, 16D was tagged by a kanamycin marker, 631 bp upstream of GLO1. As in step 1, primers were used that contain a 19 bp region binding to pJET1,2-B-KANMX-P and 50 bp tails that are homologous to the regions flanking the location where the marker needs to be inserted. The primers used for amplification of the cassette from pJET1,2-B-KANMX-P are B-2610 and B-2827.

After transformation of this fragment, selection on YPD plates containing geneticin and confirmation of the colonies by PCR with primer couple A-3863/B-2612, genomic DNA of this strain was used as a template for amplification of the tagged GLO1_16D allele. Primers B-2965 and B-2611 were used for amplification of the tagged GLO1_16D allele. (FIG. 2C)

3. Finally, the PCR product of the tagged GLO1_16D allele, containing the GLO1 allele of 16D linked to a KANMX cassette, was transformed in ER18 glo1Δ::AttL, the strain obtained after step 1. After transformation of this fragment, selection on YPD plates containing geneticin and confirmation of the colonies by PCR with primer couple A-3863/B-2612, the KANMX cassette is removed by the action of the phiC31 integrase (described previously). (FIG. 2D)

### Example 5: The GLO1 Allele from Strain 16D is Needed and Sufficient to Confer Acetic Acid Tolerance

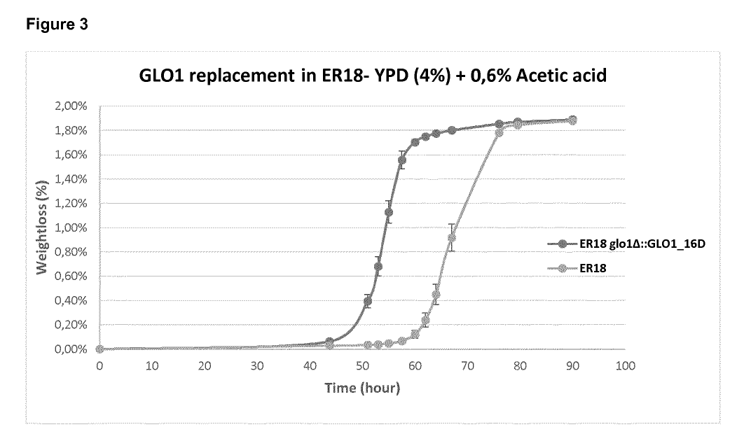

The fermentation profiles of the ER18 parental strain, and the ER18 strain in which the original GLO1 allele has been replaced by an 16D GLO1 allele are shown in FIG. 3. Every point represents the average of two biological repeats. The error bars indicate the standard error of the mean. ER18 is the original inferior parent. ER18 glo1Δ::GLO1_16D is the ER18 strain in which the GLO1 gene comprising the ORF, 631 bp upstream and 44 bp downstream of the ORF of GLO1 were replaced by its 16D counterpart.

TABLE II
Presence of non-synonymous mutations and the corresponding codons
and encoded amino acids in GLO1 from different S. cerevisiae strains
for which the whole genome sequence is available.
GLO1

| | nt | | nt | |
|---|---|---|---|---|
| | (+106-108) | aa (36) | (+964-966) | aa (322) |
| ER18 | ACC | T | CAT | H |
| 16D | GCT | A | TAT | Y |
| S288C | ACC | T | CAT | H |
| AWRI1631 | GCT | A | TAT | Y |
| AWRI796 | GCT | A | TAT | Y |
| BY4741 | ACC | T | CAT | H |
| BY4742 | ACC | T | CAT | H |
| CBS7960 | GCT | A | CAT | H |
| CEN.PK113 | ACC | T | CAT | H |
| CLIB215 | GCT | A | TAT | Y |
| EC1118 | GCT | A | TAT | Y |
| EC9-8 | GCT | A | TAT | Y |
| FL100 | ACC | T | CAT | H |
| FostersB | GCT | A | CAT | H |
| FostersO | GCT | A | CAT | H |
| JAY291 | GCT | A | CAT | H |
| Kyokai7 | ACC | T | CAT | H |
| LalvinQA23 | GCT | A | — | — |
| PW5 | ACT | T | CAT | H |
| RM11-1a | GCT | A | TAT | Y |
| Sigma1278b | ACC | T | CAT | H |
| T7 | ACT | T | CAT | H |
| UC5 | ACC | T | CAT | H |
| VL3 | — | — | TAT | Y |
| Vin13 | GCT | A | TAT | Y |
| W303 | ACC | T | CAT | H |
| YJM269 | ACC | T | CAT | H |
| YJM789 | ACT | T | CAT | H |
| ZTW1 | ACC | T | TAT | Y |

GLO1 of strain LalvinQA23 has an early stop codon resulting in a truncated ORF that lacks amongst others nt 964-965 and codes for a truncated protein that lacks amongst others aa 322.
GLO1 of strain VL3 lacks nucleotides 1-58 of the ORF and starts with the ATG at position 559-561 resulting in a shortened protein. Hence, it lacks nt 106-108 and aa 36, but not nt 964-966 and aa 322.

TABLE III
GLO1 Promoter mutations: comparison of strains

| | −782 | −775 | −645b | −645a | −562 | −559 | −531 | −460 | −431 | −385b | −385a | −384 | −273 | −230 | −219 | −135 | −77 | −64 | −48 |
|---|---|---|---|---|---|---|---|---|---|---|---|---|---|---|---|---|---|---|---|
| ER18 | A | A | — | — | T | G | C | C | A | A | T | T | C | A | C | C | — | G | T |
| 16D | G | G | C | A | C | A | C | T | G | A | T | T | T | T | T | G | A | G | — |
| S288C | A | G | — | — | C | G | T | T | G | — | — | C | C | A | C | C | A | A | T |
| AWRI1631 | G | G | C | A | C | A | C | T | G | A | T | T | T | T | T | G | A | G | — |
| AWRI796 | G | G | C | A | C | A | C | T | G | A | T | T | T | T | T | G | A | G | T |
| CBS7960 | G | G | C | A | C | A | C | T | G | A | T | T | T | A | C | G | A | G | — |
| CEN. | A | G | — | — | C | G | T | T | G | — | — | C | C | A | C | C | A | A | T |
| PK113 | | | | | | | | | | | | | | | | | | | |
| CLIB215 | G | G | C | A | C | A | C | T | G | A | T | T | T | T | T | G | A | G | — |
| EC1118 | G | G | C | A | C | A | C | T | G | A | T | T | T | T | T | G | A | G | — |
| EC9-8 | G | G | C | A | C | A | C | T | G | A | T | T | T | T | T | G | A | G | — |
| FL100 | A | G | — | — | C | A | C | T | G | A | T | T | T | T | T | G | A | A | T |
| FostersB | G | G | C | A | C | A | C | T | G | A | T | T | T | W* | Y** | G | A | G | — |
| FostersO | G | G | C | A | C | A | C | T | G | A | T | T | T | W* | Y** | G | A | G | — |
| JAY291 | G | G | C | A | C | A | C | T | G | A | T | T | T | A | C | G | A | G | — |
| Kyokai7 | A | A | — | — | T | G | C | C | G | A | T | T | C | A | C | C | A | G | T |
| LalvinQA23 | G | G | C | A | C | A | C | T | G | A | T | T | T | T | T | G | A | G | — |
| PWS | A | G | — | — | C | A | C | T | G | A | T | T | T | A | C | G | A | G | — |
| RM11-1a | G | G | C | A | C | A | C | T | G | A | T | T | T | T | T | G | A | G | — |
| Sigma1278b | A | G | — | — | C | G | T | T | G | — | — | C | C | A | C | C | A | A | T |
| T7 | A | G | — | — | C | A | C | T | G | A | T | T | T | A | C | G | A | G | — |
| UC5 | A | A | — | — | T | G | C | C | G | A | T | T | C | A | C | C | A | G | T |
| VL3 | G | G | C | A | C | A | C | T | G | A | T | T | T | T | T | G | A | G | — |
| Vin13 | G | G | C | A | C | A | C | T | G | A | T | T | T | T | T | G | A | G | — |
| W303 | A | G | — | — | C | G | T | T | G | — | — | C | C | A | C | C | A | A | T |
| YJM269 | A | G | — | — | C | G | T | T | G | — | — | C | C | A | C | C | A | G | T |
| YJM789 | A | G | — | — | C | A | C | T | G | A | T | T | T | A | C | G | A | G | — |
| ZTW1 | A | A | — | — | T | G | C | C | G | A | T | T | C | A | C | C | A | G | T |

*W: A, T or U
**Y: C, T or U

### REFERENCES

Abbott, D. A. et al. (2007). Generic and specific transcriptional responses to different weak organic acids in anaerobic chemostat cultures of Saccharomyces cerevisiae. Ferns Yeast Research 7, 819-833.

Almeida, J. R. M. et al. (2007). Increased tolerance and conversion of inhibitors in lignocellulosic hydrolysates by Saccharomyces cerevisiae. Journal og Chemical Technology and Biotechnology 82, 340-349.

Bellissimi, E., van Dijken, J. P., Pronk, J. T. & van Maris, A. J. A. (2009). Effects of acetic acid on the kinetics of xylose fermentation by an engineered, xylose-isomerase-based Saccharomyces cerevisiae strain. Ferns Yeast Research 9, 358-364.

Casey, E., Sedlak, M., Ho, N. W. & Mosier, N.S. (2010). Effect of acetic acid and pH on the cofermentation of glucose and xylose to ethanol by a genetically engineered strain of Saccharomyces cerevisiae. Ferns Yeast Research 10, 385-393.

Deutschbauer, A. M. & Davis, R. W. (2005). Quantitative trait loci mapped to single-nucleotide resolution in yeast. Nat Genet 37, 1333-1340.

Ehrenreich, I. M. et al. (2010). Dissection of genetically complex traits with extremely large pools of yeast segregants. Nature 464, 1039-1042.

Flint, J. & Mott, R. (2001). Finding the molecular basis of quantitative traits: successes and pitfalls. Nat Rev Genet 2, 437-445.

Gietz, R. D., Schiestl, R. H., Willems, A. R. & Woods, R. A. (1995). Studies on the transformation of intact yeast cells by the LiAc/SS-DNA/PEG procedure. Yeast 11, 355-360.

Glazier, A. M., Nadeau, J. H. & Aitman, T. J. (2002). Finding genes that underlie complex traits. Science 298, 2345-2349.

Hasunuma, T. et al. (2011). Metabolic pathway engineering based on metabolomics confers acetic and formic acid tolerance to a recombinant xylose-fermenting strain of Saccharomyces cerevisiae. Microb Cell Fact 10, 2.

Huang, H. et al. (2011). Identification of crucial yeast inhibitors in bio-ethanol and improvement of fermentation at high pH and high total solids. Bioresour Technol 102, 7486-7493.

Huxley C, Green E D, Dunham I. 1990. Rapid assessment of S. cerevisiae mating type by PCR. Trends Genet 6: 236.

Koppram, R., Albers, E. & Olsson, L. (2012). Evolutionary engineering strategies to enhance tolerance of xylose utilizing recombinant yeast to inhibitors derived from spruce biomass. Biotechnol Biofuels 5, 32.

Li, B. Z. & Yuan, Y. J. (2010).Transcriptome shifts in response to furfural and acetic acid in Saccharomyces cerevisiae. Appl Microbiol Biotechnol 86, 1915-1924.

Limtong, S., Sumpradit, T., Kitpreechavanich, V., Tuntirungkij, M., Seki, T. and Yoshida, T. (2000). Effect of Acetic acid on growth and ethanol fermentation of xylose fermenting yeasts and Saccharomyces cerevisiae. Kastaert J. (Nat. Sci) 34, 64-73.

Matsushika, A. & Sawayama, S. (2012). Characterization of a Recombinant Flocculent Saccharomyces cerevisiae Strain That Co-Ferments Glucose and Xylose: II. Influence of pH and Acetic Acid on Ethanol Production. Appl Biochem Biotechnol.

Mira, N. P., Palma, M., Guerreiro, J. F. & Sa-Correia, I. (2010a). Genome-wide identification of Saccharomyces cerevisiae genes required for tolerance to acetic acid. Microb Cell Fact 9, 79-91.

Mira, N. P., Becker, J. D. & Sa-Correia, I. (2010b). Genomic expression program involving the Haa1p-regulon in Saccharomyces cerevisiae response to acetic acid. OMICS 14, 587-601.

Narendranath, N. V., Thomas, K. C. & Ingledew, W. M. (2001). Effects of acetic acid and lactic acid on the growth of Saccharomyces cerevisiae in a minimal medium. J Ind Microbiol Biotechnol 26, 171-177.

Olsson, L. and Hahn-Hagerdal, B. (1993). Fermentative performance of bacteria and yeast in lignocellulose hydrolysates. Process Biochem. 28, 249-257.

Parts, L. et al. (2011). Revealing the genetic structure of a trait by sequencing a population under selection. Genome Res.

Sherman, F. & Hicks, J. (1991). Micromanipulation and dissection of asci. Methods Enzymol 194, 21-37.

Steinmetz, L. M. et al. (2002). Dissecting the architecture of a quantitative trait locus in yeast. Nature 416, 326-330.

Swinnen, S. et al. (2012). Identification of novel causative genes determining the complex trait of high ethanol tolerance in yeast using pooled-segregant whole-genome sequence analysis. Genome Res 22, 975-984.

Taherzadeh, M. J., Niklasson, C. & Lidén, G. (1997). Acetic acid—friend of foe in anaerobic batch conversion of glucose to ethanol by Saccharomyces cerevisiae. Chemical Engineering Science 52, 2653-2659.

Tanaka, K., Ishii, Y., Ogawa, J. And Shima, J. (2012). Enhancement of acetic acid tolerance in Saccharomyces cerevisiae by overexpression of the HAA1 gene, encoding a transcription factor. Appl. Environ. Microbiol. 78, 8161-8163.

Winzeler E A, Shoemaker D D, Astromoff A, Liang H, Anderson K, Andre B, Bangham R, Benito R, Boeke J D, Bussey H, et al. 1999. Functional characterization of the S. cerevisiae genome by gene deletion and parallel analysis. Science 285: 901-906.

Wright, J. et al. (2011). Batch and continuous culture-based selection strategies for acetic acid tolerance in xylose-fermenting Saccharomyces cerevisiae. Ferns Yeast Research.

Zhang, J. G. et al. (2011). Improvement of acetic acid tolerance and fermentation performance of Saccharomyces cerevisiae by disruption of the FPS1 aquaglyceroporin gene. Biotechnol Lett 33, 277-284.

1-10. (canceled)

11. A xylose fermenting yeast strain comprising a GLO1 allele, which confers on the strain improved fermentation performance in the presence of at least 0.4% acetic acid in the culture medium as compared to performance of a control strain that is genetically identical except for the GLO1 allele,
wherein the improved fermentation performance is manifest as a faster fermentation rate or a shorter lag period.

12. The yeast strain according to claim 11, wherein the improved fermentation performance occurs in the presence of at least 0.5%, acetic acid in the medium.

13. The yeast strain according to claim 11 wherein the improved fermentation occurs in the presence of at least 0.6% acetic acid in the medium.

14. The yeast strain according to claim 11 wherein the improved fermentation occurs in the presence of at least 0.7% acetic acid in the medium.

15. The yeast strain according to claim 11 wherein the improved fermentation occurs in the presence of at least 0.8% acetic acid in the medium.

16. The A xylose fermenting yeast strain according to claim 11, wherein the GLO1 allele is overexpressed.

17. The yeast strain according to claim 16, wherein the GLO1 allele is overexpressed as a result of:
(a) incorporating more than one copy of the allele in the strain, and/or
(b) the coding sequence of the allele being under control of a strong promoter.

18. The yeast strain according to claim 11 that is selected from the group consisting of Saccharomyces sp., Pichia sp., Candida sp., Pachysolen sp. and Spathaspora sp.

19. The yeast strain according to claim 18 that is a member of the species Saccharomyces cerevisiae.

20. The yeast strain according to claim 11, wherein the GLO1 allele:
(a) encodes a polypeptide the amino acid sequence of which is SEQ ID NO:2; or
(b) comprises a nucleic acid sequence which is SEQ ID NO:1.

21. A process for producing bioethanol, comprising culturing the yeast strain according to claim 11, in the presence of xylose to produce ethanol.

22. The process according to claim 21, wherein the bioethanol is produced from a hydrolysate of lignocellulose.
